(19) United States
(12) Patent Application Publication
(10) Pub. No.: US 2006/0111743 A1
(43) Pub. Date: May 25, 2006

(54) Method for arresting seizure activity

(76) Inventors: Harry C. Gurney JR., Evergreen, CO (US); Janice A. Gurney, Evergreen, CO (US)

Correspondence Address: PATTON BOGGS, L.L.P., 2001 ROSS AVENUE, SUITE 3000, DALLAS, TX, 75201, US

(21) Appl. No.: 10/993,822

(22) Filed: Nov. 19, 2004

(51) Int. Cl. A61N 1/39

(52) U.S. Cl. 607/1

(57) ABSTRACT

The method includes applying a cold thermal material to an area of the back of an individual suffering from seizure activity. The cold thermal material may be a material that is capable of providing temperatures substantially near 32° F. directly to the skin of the back of the individual suffering from seizure activity.

### FIELD OF THE INVENTION

This invention relates to a method for arresting seizures in individuals, and in particular to non-pharmacological methods for arresting seizures.

### Problem

Witnessing an individual having a seizure can be traumatic and frightening. There is a tendency for a person watching the seizure to panic, feeling helpless and hopeless during the seizure event. People often support the individual having a seizure with verbalizations, assurances, and touching. Unfortunately, people unconsciously communicate their own concern and panic, which may result in an exacerbation of the seizure episode. It is important to abbreviate seizure episodes.

These seizures can be caused by a host of diseases and illnesses, including epilepsy and many central nervous system diseases. Many methods exist today to control or arrest these seizures, but they generally involve administering strong medications to the individual or other invasive measure, such as stimulating predetermined nerves to cease the seizures. These measures and methods are generally administered by trained medical professionals.

Some of these invasive methods include, implanting a signal generator and electrodes that stimulate a particular nerve or the central nervous system in the individual. Another method involves implanting a pacemaker in the brain of an individual that detects and ameliorates seizures caused by epilepsy. Other times, non-invasive methods are used to arrest these seizures and can include using a pillow for supporting the head of a person experiencing a seizure.

Many pharmaceutical compositions and preparations exist to combat or arrest these seizure and include administering effective amounts of 4,4′-thiodianiline, 4,4′-diaminobenzophenone, 4,4′-methylenedianiline, 4,4′-diaminodiphenyl ether, or (3-aminophenyl)-(4-aminophenyl) amine, an analog, or a pharmaceutically accepted salt or complex. Other pharmaceutical compositions include comprise administering S-tofisopam substantially free of R-tofisopam to an individual. Other methods include administering preparations containing of agmatine, congeners, analogs or derivatives thereof. Still other methods include administering pharmaceutical compositions containing 2-phenyl-1,3-propanediol dicarbamate or formulations of Coenzyme Q. In addition, many tranquilizer formulations have been administered to arrest seizure activity in individuals.

Information relevant to attempts to address these problems can be found in U.S. Pat. App. Pub. Nos. US2004/0138721 filed 13 Nov. 2003 by Osorio et al.; US2003/0186942 filed 16 Jan. 2003 by Crooks et al.; US2003/0083716 filed 23 Oct. 2003 by Nicolelis.; US2003/0055048 filed 8 Nov. 2003 by Leventer et al.; US2002/0072770 filed 11 Sep. 2001 by Pless; US2002/0065323 filed 15 Jun. 2001 by Crooks et al.; and U.S. Pat. Nos. 6,649,607 issued 18 Nov. 2003 to Leventer et al.; U.S. Pat. No. 6,597,954 issued 22 Jul. 2003 to Pless; and U.S. Pat. No. 6,374,140 issued 16 Apr. 2002 to Rise; U.S. Pat. No. 6,263,876 issued 24 Jul. 2001 to Butts; U.S. Pat. No. 5,292,772 issued 8 Mar. 1994 to Sofia; U.S. Pat. No. 4,500,524 issued 19 Feb. 1985 to Catsimpoolas; and U.S. Pat. No. 4,491,594 issued 1 Jan. 1985 to Ogawa et al. However, each one of these references suffers from one or more of the following disadvantages: administration of strong medications and pharmaceuticals to individuals, invasive means of implanting electrodes, and otherwise expensive methods for arresting seizure activity.

Therefore, there is a need for a technique that efficiently and inexpensively arrests the seizures affecting individuals without the necessity of administering medications and pharmaceuticals or using electricity and implanted electrodes.

### Solution

The foregoing problems are solved and a technical advance in the art is achieved by the present method for arresting seizure activity. The method includes a simple and effective technique for abbreviating seizures of all kinds. The method involves the application of a cold thermal material to the back of the person during ictus (seizure). The cold thermal material can include block, cubed or crushed ice or other known devices that provide cold temperatures, such as commonly known ice packs or chemical cold packs.

It has been found that holding the ice on the person's back during and until the seizure subsides, results in termination of the seizure with the return to a functional and alert state common to the patient. This technique enables doctors, family, care people and individuals to be proactive in assisting a person experiencing a seizure by arresting and/or shortening the seizure and augmenting recovery.

### SUMMARY

Preferably, the present method for arresting seizure activity includes applying ice directly on the skin of the back of a person suffering from seizure activity. A benefit of this technique is that it provides the doctor, family, care person and/or individual a simple method of assisting another person who is suffering from a seizure.

These and other features, aspects, and advantages of the method for arresting seizures will become better understood with regard to the following description, appended claims, and accompanying drawings.

### BRIEF DESCRIPTION OF THE DRAWINGS

FIG. 1 illustrates in pictorial diagram form the overall method for arresting seizure activity of the present invention; and

FIG. 2 illustrates in block flow diagram the method for arresting seizure activity of the present invention.

### DETAILED DESCRIPTION OF THE DRAWINGS

The present technique for arresting seizures is directed at aborting ictus with concomitant benefits. Ictus means a seizure, stroke, blow, or sudden attack due to a condition or disease, such as epilepsy. The thermal cold material application procedure can be administered in any setting where the seizure episode may occur. Generally, cold thermal material means any device, apparatus, or material that is capable of producing temperatures at, near, or below 32° F. and that are administrable to the exposed skin of the back of a person experiencing the seizure. Some examples of thermal cold material include: ice, blocks of ice, chunks of ice, crushed ice, frozen gel packs, chemical ice packs, or objects that can maintain a temperature at, below, or near 32° F. or a temperature lower than 32° F., but that will not cause damage to the skin during its application to the skin. Preferably, the cold thermal material is capable of producing temperatures in the range of from about 0° F. to about between 50° F. More preferably, the cold thermal material is capable of producing temperatures in the range of from about 20° F. to about 40° F. Most preferably, the cold thermal material is capable of producing temperatures at or near 32° F.

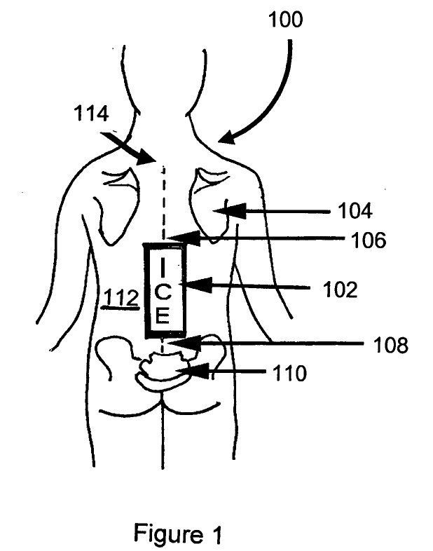

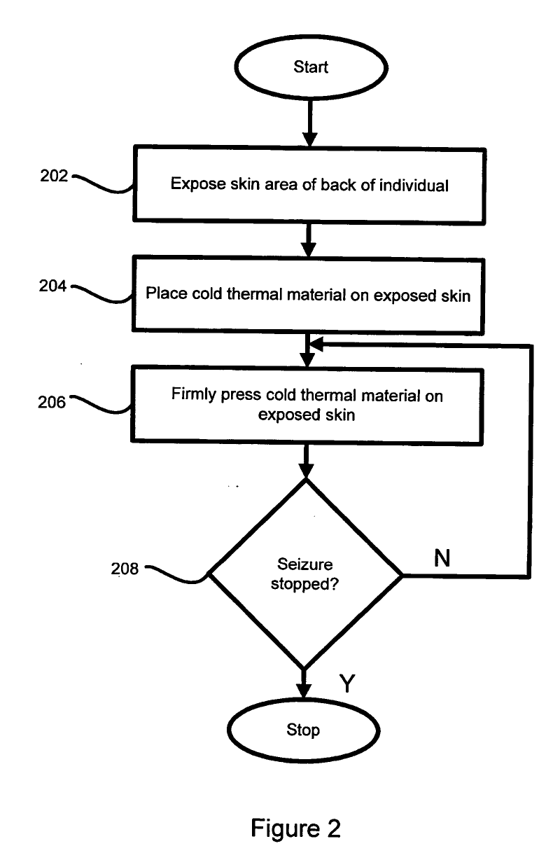

FIGS. 1 and 2 depict an exemplary embodiment of the present method for arresting seizure activity in an individual 100. In this embodiment, the skin 112 of an individual 100 is exposed 202 for treatment. A cold thermal material 102, such as ice, is applied 204 to the exposed skin 112 of an individual 100 suffering from seizure activity as soon as the seizure is observed. In this embodiment, the cold thermal material 102 is a block of ice (i.e., water frozen in an ice tray or rectangular plastic container) and the ice is applied directly to the skin 112 of the individual 100 suffering from the seizure over a specified area on the individual's back 114. The ice is held firmly 206 to the individual's back 114, on an area superior to the spinal process from Thoracic 10 (T10) 106 to Lumbar 4 (L4) 108. This area is approximately two inches below the lower tip of the scapula 104, three inches above the sacrum 110, and three inches to the left and right of the center of the spine as illustrated in FIG. 1. The area of a given ice application should be sufficient to cover the described area of the skin 112 of the back 114 of an individual 100 suffering from seizure activity. The ice is pressed firmly 206 against the skin 112 of the back 114 of the individual 100 and constant pressure is applied on the back 114 until the seizure is stopped 208 and the individual 100 recovers from the ictus and makes efforts to sit or otherwise change position due to the alleviation of the ictus.

Generally, the sooner the cold thermal material 102 is applied during a seizure event, the more effective and immediate the intervention is in arresting or abbreviating the seizure. It has been noted that the post-ictus recovery time is shorter and recovery appears augmented when prompt use of the technique is employed. Application of cold thermal material 102 either terminates or significantly abbreviates ictus and post-ictus duration. This technique is considered helpful for seizures of any origin, including cancers of the central nervous system.

If the individual is prone to “chain” episodes or displays evidence of returning to ictus after removal of the cold thermal material 102, the cold thermal material 102 should once again be applied until the individual 100 regains a conscious state.

Although there has been described what is at present considered to be the preferred embodiments of the present invention, it will be understood that the invention can be embodied in other specific forms without departing from the spirit or essential characteristics thereof. For example, there may exist other portions of the body where the application of the cold thermal material may also be effective in arresting seizure activity. The present embodiments are, therefore, to be considered in all aspects as illustrative and not restrictive. The scope of the invention is indicated by the appended claims rather than the foregoing description.

What is claimed:

1. A method for arresting seizure activity in an individual, comprising:
applying a cold thermal material to an area of a back of said person during said seizure activity until said seizure activity ceases.

2. The method for arresting seizure activity of claim 1, wherein said area of said back comprises:
an area superior to the spinal process from T10 to L4 of said individual.

3. The method for arresting seizure activity of claim 1, wherein said area of said back comprises:
an area approximately two inches below the lower tip of the scapula, three inches above the sacrum, and three inches to the left and right of the center of the spine of said individual.

4. The method for arresting seizure activity of claim 1, wherein said applying comprises:
applying the cold thermal material directly to the skin of said back of said individual.

5. The method for arresting seizure activity of claim 1 wherein said applying comprises:
pressing said cold thermal material firmly against said back of said person experiencing said seizure activity.

6. The method for arresting seizure activity of claim 1 wherein said cold thermal material produces temperatures in the range of from about 0° F. to about between 50° F.

7. The method for arresting seizure activity of claim 1 wherein said cold thermal material produces temperatures in the range of from about 20° F. to about 40° F.

8. The method for arresting seizure activity of claim 1 wherein said cold thermal material produces temperatures at or substantially near 32° F.

9. The method for arresting seizure activity of claim 1 wherein said cold thermal material is selected from the group consisting of ice, ice cold compresses, ice thermal packs, and chemical ice packs.

10. The method for arresting seizure activity of claim 1 wherein said cold thermal material is ice.
